(19) United States
(12) Patent Application Publication
(10) Pub. No.: US 2002/0040030 A1
(43) Pub. Date: Apr. 4, 2002

(54) Alpha V integrin receptor antagonists

(76) Inventors: Paul J. Coleman, Wallingford, PA (US); Donghui Cui, Newtown, PA (US); Mark E. Duggan, Schwenksville, PA (US); John H. Hutchinson, Philadelphia, PA (US); Thomayant Prueksaritanont, Landsdale, PA (US); Maria Victoria Silva Elipe, Mountainside, NJ (US); Xiaojun Fang, Kalamazoo, MI (US)

Correspondence Address: MERCK AND CO INC, P O BOX 2000, RAHWAY, NJ, 070650907

(21) Appl. No.: 09/953,606

(22) Filed: Sep. 14, 2001

Related U.S. Application Data
(60) Provisional application No. 60/232,262, filed on Sep. 14, 2000.

(51) Int. Cl. C 07D 4 3/14; A61K 31/506

(52) U.S. Cl. 514/256; 544/333

(57) ABSTRACT

The present invention relates to novel compounds formed by metabolic conversion of compounds of the structural formula depicted below (R═H or Me), pharmaceutical compositions containing such compounds, and their use as αvβ3 integrin receptor antagonists. The compounds of the present invention are useful for inhibiting bone resorption, restenosis, angiogenesis, diabetic retinopathy, macular degeneration, inflammatory arthritis, cancer, and metastatic tumor growth. They are particularly useful for inhibiting bone resorption and for the treatment and prevention of osteoporosis.

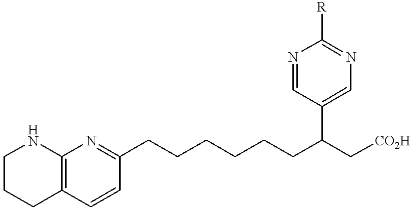

### CROSS-REFERENCE TO RELATED APPLICATIONS

The present invention is related to U.S. provisional application Serial No. 60/232,262, filed Sep. 14, 2000, the contents of which are hereby incorporated by reference.

### BACKGROUND OF THE INVENTION

U.S. Pat. No. 6,048,861 (issued Apr. 11, 2000) discloses compounds of structural formulae (1) and (2):

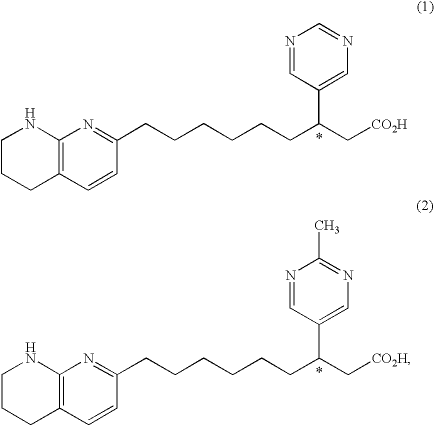

which include the two enantiomeric forms at the C-3 position (marked with *)of the nonanoic acid chain.

These compounds are antagonists of the integrin receptor αvβ3 and are therefore useful for inhibiting bone resorption, restenosis, angiogenesis, diabetic retinopathy, macular degeneration, inflammatory arthritis, cancer, and metastatic tumor growth. They are particularly useful for inhibiting bone resorption and for the treatment and prevention of osteoporosis.

### SUMMARY OF THE INVENTION

The present invention relates to derivatives of 3-(pyrimidin-5-yl)- and 3-(2-methyl-pyrimidin-5-yl)-9-(5,6,7,8-tetrahydro[1,8]naphthyridin-2-yl)-nonanoic acid (1) and (2), respectively, methods for their preparation, pharmaceutical compositions containing such compounds, and methods for using these compounds as αvβ3 integrin receptor antagonists. These derivatives are formed by metabolic conversion of the compounds of formulae (1) and (2).

Because of their activity as αvβ3 integrin receptor antagonists, the compounds of the present invention are useful, inter alia, for inhibiting bone resorption and for the treatment and prevention of osteoporosis.

### DETAILED DESCRIPTION OF THE INVENTION

The present invention provides compounds of structural formula I:

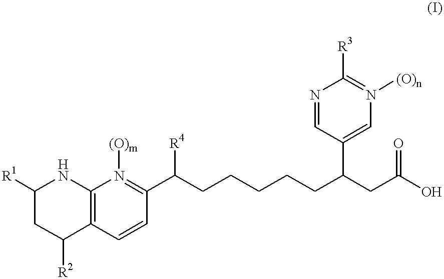

wherein

R¹, R², and R⁴ are each independently hydrogen, hydroxy, or oxo;

R³ is hydrogen or methyl;

m is 0 or 1;

n is 0or 1;

provided that at least one of R¹, R², and R⁴ is hydroxy or oxo or at least one of n or m is 1;

and the individual stereoisomers thereof, or a pharmaceutically acceptable salt thereof.

One embodiment of the present invention is directed to compounds of structural formula II:

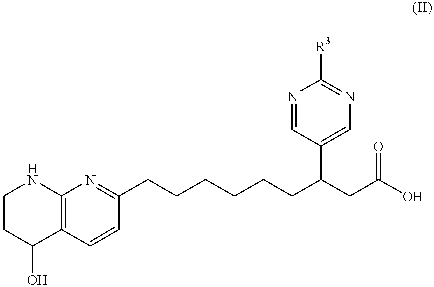

wherein R³ is hydrogen or methyl;

and the individual stereoisomers thereof, or a pharmaceutically acceptable salt thereof.

One class of this embodiment is directed to compounds of structural formula III:

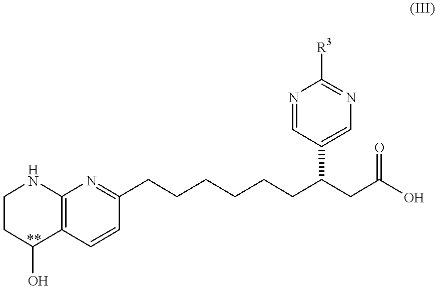

wherein R³ is hydrogen or methyl;

and the stereoisomers thereof at the hydroxylated C-5 position (marked with **) of the tetrahydro-[1,8]naphthyridine ring;

or a pharmaceutically acceptable salt thereof.

A second embodiment of the present invention is directed to compounds of structural formula IV:

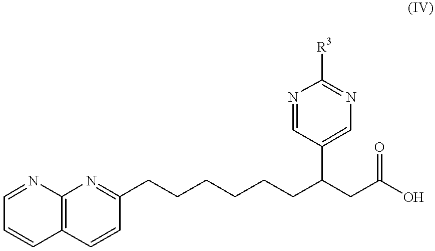

wherein R³ is hydrogen or methyl;

and the individual stereoisomers thereof, or a pharmaceutically acceptable salt thereof.

One class of this embodiment is directed to compounds of structural formula V:

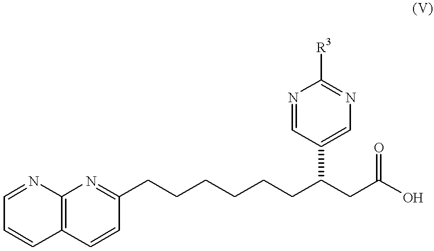

wherein R³ is hydrogen or methyl;

or a pharmaceutically acceptable salt thereof.

A third embodiment of the present invention is directed to compounds of structural formula VI:

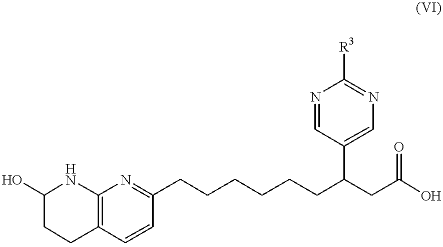

wherein R³ is hydrogen or methyl;

and the individual stereoisomers thereof, or a pharmaceutically acceptable salt thereof.

One class of this embodiment is directed to compounds of structural formula VII:

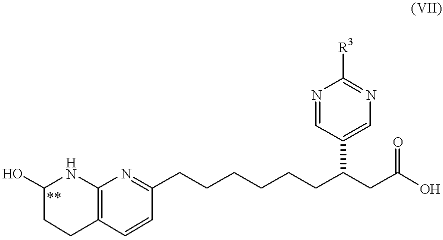

wherein R³ is hydrogen or methyl;

and the stereoisomers thereof at the hydroxylated C-7 position (marked with **) of the tetrahydro-[1,8]naphthyridine ring;

or a pharmaceutically acceptable salt thereof.

A fourth embodiment of the present invention is directed to compounds of structural formula VIII:

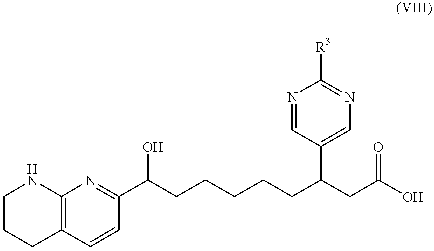

wherein R³ is hydrogen or methyl;

and the individual stereoisomers thereof, or a pharmaceutically acceptable salt thereof.

One class of this embodiment is directed to compounds of structural formula IX:

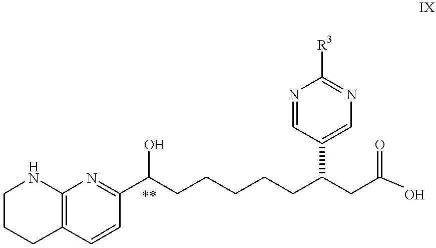

wherein R³ is hydrogen or methyl;

and the stereoisomers thereof at the hydroxylated C-9 position (marked with **) of the nonanoic acid chain;

or a pharmaceutically acceptable salt thereof.

A fifth embodiment of the present invention is directed to compounds of structural formula X:

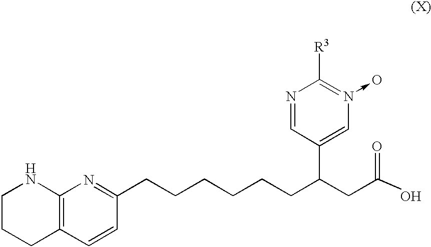

wherein R³ is hydrogen or methyl;

and the individual stereoisomers thereof, or a pharmaceutically acceptable salt thereof.

One class of this embodiment is directed to compounds of structural formula XI:

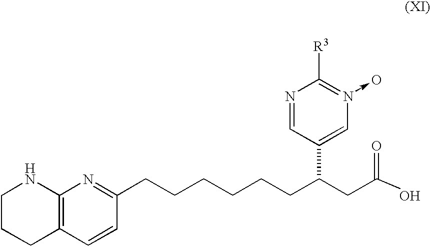

wherein R³ is hydrogen or methyl;

or a pharmaceutically acceptable salt thereof.

A sixth embodiment of the present invention is directed to compounds of structural formula XII:

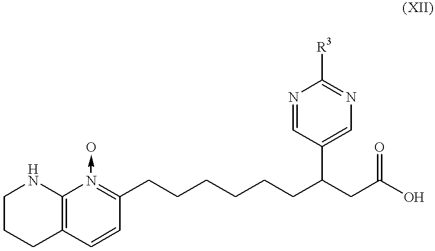

wherein R³ is hydrogen or methyl;

and the individual stereoisomers thereof, or a pharmaceutically acceptable salt thereof.

One class of this embodiment is directed to compounds of structural formula XIII:

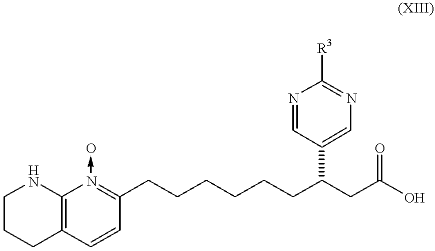

wherein R³ is hydrogen or methyl;

or a pharmaceutically acceptable salt thereof.

For use in medicine, the salts of the compounds of this invention refer to non-toxic “pharmaceutically acceptable salts.” Other salts may, however, be useful in the preparation of the compounds according to the invention or of their pharmaceutically acceptable salts. Salts of basic compounds encompassed within the term “pharmaceutically acceptable salts” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The compounds of the present invention can have chiral centers and can thus occur as racemates, racemic mixtures, single enantiomers, diastereomeric mixtures, and individual diastereomers, with all isomeric forms being included in the present invention. Therefore, where a compound is chiral, the separate enantiomers or diastereomers, substantially free of the other, are included within the scope of the invention; further included are all mixtures of the two enantiomers.

Compounds of the present invention may be separated into enantiomeric pairs of diastereoisomers by, for example, fractional crystallization from a suitable solvent, for example, methanol or ethyl acetate or a mixture thereof. The pair of enantiomers thus obtained may be separated into individual stereoisomers by conventional means, for example, by the use of an optically active acid as a resolving agent, or by HPLC using a chiral stationary phase. Alternatively, any steroisomer of a compound of the present invention may be obtained by stereospecific synthesis using optically pure starting materials or reagents of known configuration.

Also included within the scope of the invention are polymorphs and hydrates of the compounds of the instant invention.

The term “therapeutically effective amount” shall mean that amount of a drug or pharmaceutical agent that will elicit the biological or medical response of a tissue, system, animal or human that is being sought by a researcher or clinician.

The term “integrin receptor antagonist,” as used herein, refers to a compound which binds to and antagonizes the αvβ3 receptor.

The term “bone resorption,” as used herein, refers to the process by which osteoclasts degrade bone.

Compounds of the present invention typically display an affinity for the αvβ3 integrin receptor of less than 100 nanomolar. Compounds of this invention are therefore useful for treating mammals suffering from a bone condition caused or mediated by increased bone resorption, who are in need of such therapy. Pharmacologically effective amounts of the compounds, including pharmaceutically acceptable salts thereof, are administered to the mammal, to inhibit the activity of mammalian osteoclasts.

The compounds of the present invention are administered in dosages effective to antagonize the αvβ3 receptor where such treatment is needed, as, for example, in the prevention or treatment of osteoporosis.

Illustrating the invention is the method for eliciting an αvβ3 antagonizing effect. More particularly, the αvβ3 antagonizing effect is selected from inhibition of: bone resorption, restenosis, angiogenesis, diabetic retinopathy, macular degeneration, inflammation, inflammatory arthritis, viral disease, cancer, and metastatic tumor growth. In one embodiment of the method, the αvβ3 antagonizing effect is the inhibition of bone resorption.

More particularly illustrating the invention is a pharmaceutical composition comprising any of the compounds described above and a pharmaceutically acceptable carrier. Another example of the invention is a pharmaceutical composition made by combining any of the compounds described above and a pharmaceutically acceptable carrier. Another illustration of the invention is a process for making a pharmaceutical composition comprising combining any of the compounds described above and a pharmaceutically acceptable carrier.

Further illustrating the invention is a method of treating and/or preventing a condition mediated by antagonism of the αvβ3 integrin receptor in a mammal in need thereof, comprising administering to the mammal a therapeutically effective amount of any of the compounds described above. Preferably, the condition is selected from bone resorption, osteoporosis, restenosis, diabetic retinopathy, macular degeneration, angiogenesis, atherosclerosis, inflammation, inflammatory arthritis, viral disease, cancer, and metastatic tumor growth. More preferably, the condition is selected from osteoporosis and cancer. Most preferably, the condition is osteoporosis.

More specifically exemplifying the invention is a method of eliciting an αvβ3 integrin antagonizing effect in a mammal in need thereof, comprising administering to the mammal a therapeutically effective amount of any of the compounds or any of the pharmaceutical compositions described above. Preferably, the αvβ3 antagonizing effect is selected from inhibition of bone resorption, inhibition of restenosis, inhibition of atherosclerosis, inhibition of angiogenesis, inhibition of diabetic retinopathy, inhibition of macular degeneration, inhibition of inflammation, inhibition of viral disease, or inhibition of cancer or metastatic tumor growth. More preferably, the αvβ3 antagonizing effect is inhibition of bone resorption.

Additional examples of the invention are methods of inhibiting bone resorption and of treating and/or preventing osteoporosis in a mammal in need thereof, comprising administering to the mammal a therapeutically effective amount of any of the compounds or any of the pharmaceutical compositions decribed above.

Additional illustrations of the invention are methods of treating hypercalcemia of malignancy, osteopenia due to bone metastases, periodontal disease, hyperparathyroidism, periarticular erosions in rheumatoid arthritis, Paget's disease, immobilization-induced osteopenia, and glucocorticoid treatment in a mammal in need thereof, comprising administering to the mammal a therapeutically effective amount of any of the compounds or any of the pharmaceutical compositions described above.

More particularly exemplifying the invention is the use of any of the compounds described above in the preparation of a medicament for the treatment and/or prevention of osteoporosis in a mammal in need thereof. Still further exemplifying the invention is the use of any of the compounds described above in the preparation of a medicament for the treatment and/or prevention of bone resorption, cancer, metastatic tumor growth, restenosis, atherosclerosis, diabetic retinopathy, macular degeneration, inflammation, inflammatory arthritis, viral disease, and/or angiogenesis.

Also exemplifying the invention are compositions further comprising an active ingredient selected from the group consisting of

a) an organic bisphosphonate or a pharmaceutically acceptable salt or ester thereof,

b) an estrogen receptor modulator,

c) an androgen receptor modulator,

d) a cytotoxic/antiproliferative agent,

e) a matrix metalloproteinase inhibitor,

f) an inhibitor of epidermal-derived, fibroblast-derived, or platelet-derived growth factors,

g) an inhibitor of VEGF,

h) an antibody to a growth factor or to a growth factor receptor,

i) an inhibitor of Flk-1/KDR, Flt-1, Tck/Tie-2, or Tie-1,

j) a cathepsin K inhibitor,

k) a growth hormone secretagogue,

l) an inhibitor of osteoclast proton ATPase,

m) an inhibitor of urokinase plasminogen activator (u-PA),

n) a tumor-specific antibody-interleukin-2 fusion protein,

o) an inhibitor of HMG-CoA reductase, and

p) a farnesyl transferase inhibitor or a geranylgeranyl transferase inhibitor or a dual farnesyl/geranylgeranyl transferase inhibitor; and mixtures thereof.

(See, B. Millauer et al., “Dominant-Negative Inhibition of Flk-1 Suppresses the Growth of Many Tumor Types in Vivo”, Cancer Research, 56, 1615-1620 (1996), which is incorporated by reference herein in its entirety).

Preferably, the active ingredient is selected from the group consisting of:

a) an organic bisphosphonate or a pharmaceutically acceptable salt or ester thereof,

b) an estrogen receptor modulator,

c) an androgen receptor modulator,

d) an inhibitor of osteoclast proton ATPase,

e) an inhibitor of HMG-CoA reductase, and

f) a cathepsin K inhibitor; and mixtures thereof.

Nonlimiting examples of such bisphosphonates include alendronate, etidronate, pamidronate, risedronate, ibandronate, and pharmaceutically acceptable salts and esters thereof. A particularly preferred bisphosphonate is alendronate, especially alendronate monosodium trihydrate.

Nonlimiting examples of estrogen receptor modulators include estrogen, progesterin, estradiol, droloxifene, raloxifene, and tamoxifene.

Nonlimiting examples of cytotoxic/antiproliferative agents are taxol, vincristine, vinblastine, and doxorubicin.

Cathepsin K, formerly known as cathepsin O2, is a cysteine protease and is described in PCT International Application Publication No. WO 96/13523, published May 9, 1996; U.S. Pat. No. 5,501,969, issued Mar. 3, 1996; and U.S. Pat. No. 5,736,357, issued Apr. 7, 1998, all of which are incorporated by reference herein in their entirety. Cysteine proteases, specifically cathepsins, are linked to a number of disease conditions, such as tumor metastasis, inflammation, arthritis, and bone remodeling. At acidic pH's, cathepsins can degrade type-I collagen. Cathepsin protease inhibitors can inhibit osteoclastic bone resorption by inhibiting the degradation of collagen fibers and are thus useful in the treatment of bone resorption diseases, such as osteoporosis.

Members of the class of HMG-CoA reductase inhibitors, known as the “statins,” have been found to trigger the growth of new bone, replacing bone mass lost as a result of osteoporosis (see The Wall Street Journal, Friday, Dec. 3, 1999, page B1). Therefore, the statins hold promise for the treatment of bone resorption. Nonlimiting examples of statins are lovastatin, simvastatin, atorvastatin, and pravastatin.

Evidence for crucial role of the urokinase-urokinase receptor (u-PA-u-PAR) in angiogenesis, tumor invasion, inflammation, and matrix remodeling during wound healing and development has been presented [see Y. Koshelnick et al., “Mechanisms of signaling through Urokinase Receptor and the Cellular Response,” Thrombosis and Haemostasis 82: 305-311 (1999) and F. Blasi, “Proteolysis, Cell Adhesion, Chemotaxis, and Invasiveness Are Regulated by the u-PA-u-PAR-PAI-1 System,” Thrombosis and Haemostasis 82: 298-304 (1999)]. Thus, specific antagonists of the binding of u-PA to u-PAR have been found to inhibit cell-surface plasminogen activation, tumor growth, and angiogenesis in both in vitro and in vivo models.

H. N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth.

The proton ATPase which is found on the apical membrane of the osteoclast has been reported to play a significant role in the bone resorption process. Therefore, this proton pump represents an attractive target for the design of inhibitors of bone resorption which are potentially useful for the treatment and prevention of osteoporosis and related metabolic diseases (see C. Farina et al., “Selective inhibitors of the osteoclast vacuolar proton ATPase as novel bone antiresorptive agents,” DDT, 4: 163-172 (1999)).

Evidence has been presented that androgenic steroids play a physiological role in the development of bone mass in men and women and that androgens act directly on bone. Androgen receptors have been demonstrated in human osteoblast-like cell lines and androgens have been shown to directly stimulate bone cell proliferation and differentiation. For a discussion, reference is made to S. R. Davis, “The therapeutic use of androgens in women,” J. Steroid Biochem. Mol. Biol., 69: 177-184 (1999) and K. A. Hansen and S. P. T. Tho, “Androgens and Bone Health,” Seminars in Reproductive Endocrinology,” 16: 129-134 (1998). Thus, androgen receptor modulators may have utility in the treatment and prevention of bone loss in women.

Activators of the peroxisome proliferator-activated receptor-γ(PPARγ), such as the thiazolidinediones (TZD's), inhibit osteoclast-like cell formation and bone resorption in vitro. Results reported by R. Okazaki et al. in Endocrinology, 140: 5060-5065 (1999) point to a local mechanism on bone marrow cells as well as a systemic one on glucose metabolism. Nonlimiting examples of PPARγ activators include troglitazone, pioglitazone, rosiglitazone, and BRL 49653.

The present invention is also directed to combinations of the compounds of the present invention with one or more agents useful in the prevention or treatment of osteoporosis. For example, the compounds of the instant invention may be effectively administered in combination with effective amounts of other agents such as an organic bisphosphonate, an estrogen receptor modulator, an androgen receptor modulator, a growth hormone secretagogue, a cathepsin K inhibitor, an HMG-CoA reductase inhibitor, a PPARγ activator, or an inhibitor of the osteoclast proton ATPase.

Additional illustrations of the invention are methods of treating tumor growth or metastasis in a mammal in need thereof, comprising administering to the mammal a therapeutically effective amount of a compound described above and one or more agents known to be cytotoxic/antiproliferative. Also, the compounds of the present invention can be administered in combination with radiation therapy for treating cancer and metastatic tumor growth.

In addition, the integrin αvβ3 antagonist compounds of the present invention may be effectively administered in combination with a growth hormone secretagogue in the therapeutic or prophylactic treatment of disorders in calcium or phosphate metabolism and associated diseases. These diseases include conditions which can benefit from a reduction in bone resorption. A reduction in bone resorption should improve the balance between resorption and formation, reduce bone loss or result in bone augmentation. A reduction in bone resorption can alleviate the pain associated with osteolytic lesions and reduce the incidence and/or growth of those lesions. These diseases include: osteoporosis (including estrogen deficiency, immobilization, glucocorticoid-induced and senile), osteodystrophy, Paget's disease, myositis ossificans, Bechterew's disease, malignant hypercalcemia, metastatic bone disease, periodontal disease, cholelithiasis, nephrolithiasis, urolithiasis, urinary calculus, hardening of the arteries (sclerosis), arthritis, bursitis, neuritis and tetany. Increased bone resorption can be accompanied by pathologically high calcium and phosphate concentrations in the plasma, which would be alleviated by this treatment. Similarly, the present invention would be useful in increasing bone mass in patients with growth hormone deficiency. Thus, preferred combinations are simultaneous or alternating treatments of an αvβ3 receptor antagonist of the present invention and a growth hormone secretagogue, optionally including a third component comprising an organic bisphosphonate, preferably alendronate monosodium trihydrate.

In accordance with the method of the present invention, the individual components of the combination can be administered separately at different times during the course of therapy or concurrently in divided or single combination forms. The instant invention is therefore to be understood as embracing all such regimes of simultaneous or alternating treatment, and the term “administering” is to be interpreted accordingly. It will be understood that the scope of combinations of the compounds of this invention with other agents useful for treating integrin-mediated conditions includes in principle any combination with any pharmaceutical composition useful for treating osteoporosis.

As used herein, the term “composition” is intended to encompass a product comprising the specified ingredients in the specified amounts, as well as any product which results, directly or indirectly, from combination of the specified ingredients in the specified amounts.

The compounds of the present invention can be administered in such oral dosage forms as tablets, capsules (each of which includes sustained release or timed release formulations), pills, powders, granules, elixirs, tinctures, suspensions, syrups and emulsions. Likewise, they may also be administered in intravenous (bolus or infusion), intraperitoneal, topical (e.g., ocular eyedrop), subcutaneous, intramuscular or transdermal (e.g., patch) form, all using forms well known to those of ordinary skill in the pharmaceutical arts. An effective but non-toxic amount of the compound desired can be employed as an αvβ3 antagonist.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

Methods of Preparation

Compounds of formula I are biliary metabolites of the compounds of formulae (1) and (2). They can be obtained in vitro by incubation with rat, dog, monkey, or human liver microsomes or by incubation with rat, dog, monkey, or human hepatocytes. They can also be isolated and purified from the bile of individuals who have ingested compounds of formula (1) or (2), using methodologies that are well-known in the art, such as reverse-phase high-performance liquid chromatography.

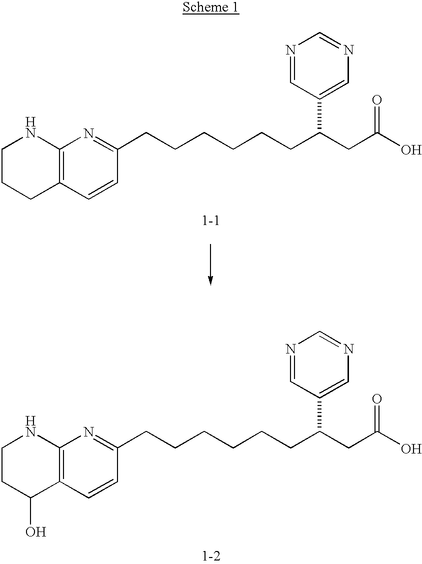

### EXAMPLE 1

### 3(S)-(Pyrimidin-5-yl)-9-[5(R or S)-hydroxy-5,6,7,8-tetrahydro[1,8]naphthyridin-2-yl]-nonanoic acid (1-2)

3(S)-(Pyrimidin-5-yl)-9-(5,6,7,8-tetrahydro[1,8]naphthyridin-2-yl)-nonanoic acid (1-1) (for preparation of 1-1, see U.S. Pat. No. 6,048,861, which is incorporated by reference herein in its entirety) (25 μM) was incubated with rat liver microsomes or rat liver S9 (1 mg proteins/mL) at 37° C. for 2.5 hours in the presence of 10 mM MgCl₂, 1 mM NADPH, and 50 mM pH 7.4 Tris buffer. The incubate was precipitated with acetonitrile containing 2% formic acid, then centrifuged to get rid of precipitates. The resulting supernatant was dried under a stream of nitrogen, then reconstituted in 0.1% formic acid containing 10% acetonitrile. The reconstituted sample was subjected to separation by high-performance liquid chromatography (HPLC) on a Hypersil C18 column (150×4.6 mm, 5 micron particle size) with the following gradient as the mobile phase: solvent A=5 mM ammonium acetate, pH 4.5; solvent B=0.1% formic acid in acetonitrile; gradient: 0-2.5-12.5-16-16.1-17.5 min. @ 10-10-30-30-80-80% of solvent B; flow rate was 2 ml/min. Compound 1-2 eluted at about 8.5 min., which was collected and further purified using the following

HPLC conditions:

Column: Eclipse XDB-C18, 250×4.6 mm

Solvent A: 0.1% TFA in water

Solvent B: 0.1% TFA in acetonitrile

Gradient: 0-5-5.2-25.2 min @ 10-10-20-20% of solvent B

Flow rate: 1 mL/min.

Compound 1-2 eluted out at about 11 minutes and was characterized by means of mass and NMR spectroscopy:

¹H NMR (400 MHz, D₂O): δ8.99 (s, 1H); 8.70 (s, 2H); 7.81 (d, 1H, J=7.4); 6.66 (d, 1H, J=7.4); 4.90 (t, 1H, J=4.5); 3.54 (m, 2H); 3.11 (m, 1H); 2.68 (t, 2H, J=7.5); 2.68 (m, 1H); 2.53 (dd, 1H, J=8.7, 14.7); 2.01 (m, 2H); 1.74 (m, 2H); 1.62 (m, 2H); and 1.29 ( m, 6H).

MS/MS spectrum: 367 (M−H₂O).

Mass spectrum: 385 (MH⁺).

Compounds 1-3 and 1-4 were also identified as metabolites of compound 1-1 by means of comparison of their liquid chromatographic mass spectra and NMR spectra with those of authentic materials prepared by unambiguous chemical synthesis detailed in the Schemes below.

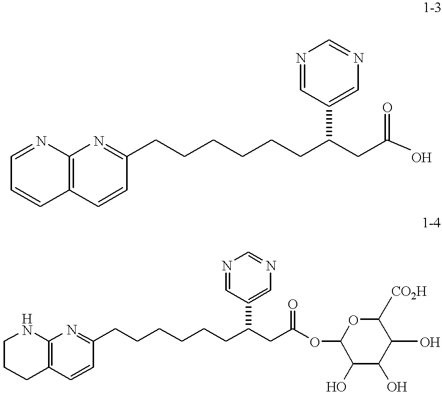

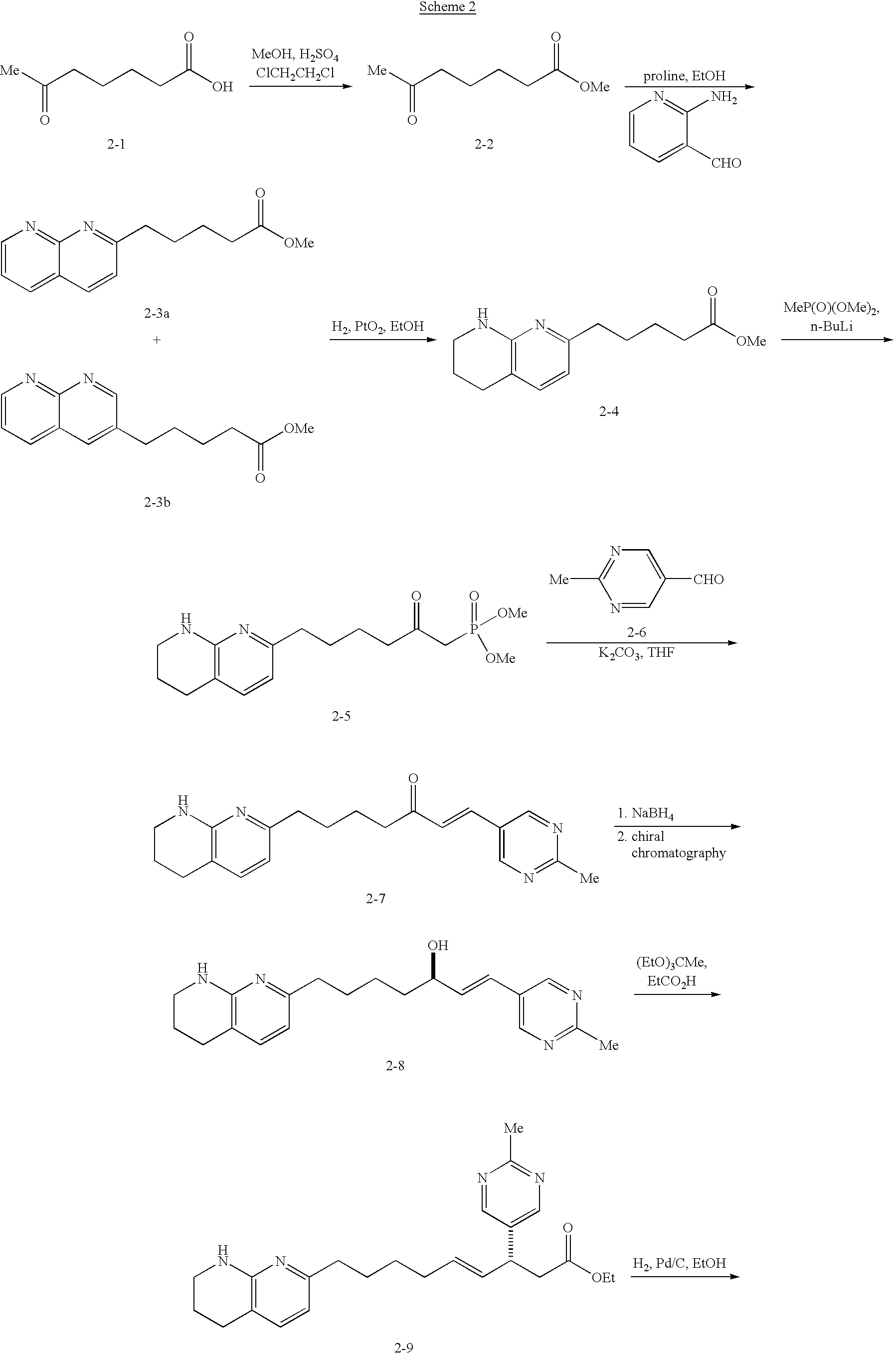

### EXAMPLE 2

### 3(S)-(2-Methyl-pyrimidin-5-yl)-9-[5(R or S)-hydroxy-5,6,7,8-tetrahydro-[1,8]naphthyridin-2-yl]-nonanoic acid (2-12)

### 6-Oxo-heptanoic acid methyl ester (2-2)

A solution of 5-acetylvaleric acid 2-1 (200 g, 1.38 mole) in 1,2-dichloroethane/methanol (1000 mL/400 mL) was treated with concentrated sulfuric acid (3.69 mL, 6.78 g, 69 mmol) and refluxed for 12 h. The solution was cooled to room temperature and washed with saturated aqueous NaHCO₃ (2×400 mL). The aqueous washes were back extracted with dichloromethane (200 mL), and the combined organic extracts were washed with brine. The solution was dried over MgSO₄, filtered, and concentrated to give about 218 g of the methyl ester 2-2 as a colorless oil.

¹H NMR (300 MHz, CDCl₃): δ3.62 (s, 3H), 2.41 (t, 2H), 2.31 (t, 3H), 2.12 (s, 3H), 1.59 (m, 4H) ppm.

### 5-[1,8]naphthyridin-2-yl-pentanoic acid methyl ester (2-3a)

A solution of the unpurified methyl ketone 2-2 (218 g, 1388 mmol), 2-amino-3-formylpyridine (169.0 g, 1388 mmol) and proline (79.8 g, 694 mmol) in anhydrous ethanol (2000 mL) was heated at reflux for 12 h. The solution was cooled to room temperature and concentrated. The residue was taken up in ethyl acetate (1000 mL) and washed with water (2×300 mL) to remove the proline. The organic solution was dried over MgSO₄, filtered and concentrated. The residue was not further purified to give 320 g of a 3:1 mixture of the desired product 2-3a and naphthyridine isomer 2-3b. A small portion of this mixture was purified by flash chromatography (SiO₂; 10% EtOH/CH₂Cl₂) to provide pure 2-3a for analysis.

¹H NMR (300 MHz, CDCl₃): δ9.06 (dd, J=2.1, 4.3 Hz, 1H), 8.14 (dd, J=2.0, 8.0 Hz, 1H), 8.09 (d, J=8.2 Hz, 1H), 7.43 (dd, J=4.3, 8.2 Hz, 1H), 7.37 (d, J=8.2 Hz 1H), 3.64 (s, 3H), 3.05 (t, J=7.6 Hz, 2H), 2.37 (t, J=7.6 Hz, 2H), 1.93 (m, 2H), 1.75 (m, 2H) ppm.

### 5-(5,6,7,8-tetrahydro-[1,8]naphthyridin-2-yl)-pentanoic acid methyl ester (2-4)

A solution of the mixture of naphthyridines 2-3a and 2-3b (160.0 g, 656 mmol) in anhydrous ethanol (2000 mL) was degassed with argon gas for 20 min. The solution was charged with PtO₂ (16.0 g) and the heterogeneous mixture placed under 1 atm of hydrogen gas and stirred for 48 h. The mixture was purged with argon, filtered through Celite and concentrated to give a dark oil. The residue was purified by flash chromatography (SiO₂, 100% EtOAc) to give an orange waxy solid which was recrystallized from diethyl ether to give 56.0 g of the desired product 2-4 as a tan crystalline solid.

¹H NMR (300 MHz, CDCl₃): δ7.04 (d, J=7.3 Hz, 1H), 6.32 (d, J=7.3 Hz, 1H), 4.73 (br s, 1H), 3.65 (s, 3H), 3.38 (m, 2H), 2.68 (t, J=6.1 Hz, 2H), 2.54 (m, 2H), 2.33 (m, 2H), 1.89 (m, 2H), 1.67 (m, 4H) ppm.

### [2-oxo-6-(5,6,7,8-tetrahydro-[1,8]naphthyridin-2-yl)-hexyl]-phosphonic acid dimethyl ester (2-5)

A solution of dimethylmethylphosphonate (112 mL, 1030 mmol) in TMF (1300 mL) was cooled to −78° C. A solution of n-BuLi (412 mL of a 2.5 M soln in hexanes, 1030 mmol) was added via an addition funnel over 1 h. The reaction became heterogeneous at the conclusion of the addition. A solution of methyl ester 2-4 (64.0 g, 258 mmol) in THF (400 mL) was added via an addition funnel over 45 min. After stirring for an additional 20 min at −78° C., the mixture was quenched with saturated aqueous NH₄Cl and the cold bath removed. After warming to room temperature, the mixture was diluted with EtOAc and washed with H₂O and brine. The organic solution was dried over MgSO₄, filtered and concentrated. The residue was not further purified to give 80.1 g of the ketophosphonate 2-5 as a pale yellow oil.

¹H NMR (400 MHz, CDCl₃): δ6.97 (d, J=7.6 Hz, 1H), 6.26 (d, J=7.6 Hz, 1H), 4.7 (br s, 1H), 3.72 (s, 3H), 3.70 (s, 3H), 3.32 (m, 1H), 3.01 (d, J=22.8 Hz, 2H), 2.59 (m, 4H), 2.46 (m, 2H), 1.83 (m, 2H), 1.57 (m, 4H) ppm.

### 2-Methyl-pyrimidine-5-carboxaldehyde (2-6)

To a solution of bromoacetic acid (12 g, 86.4 mmol) in DMF (44 mL) at 90° C. was added phosphorous oxychloride (24 mL, 260 mmol) over 5 h and then heated to 110° C. After stirring at 110° C. for 2.5 h, the mixture was cooled to 45° C. and quenched into a cold isopropanol (44 mL) at 2° C. and diluted with isopropyl acetate (44 mL) and then treated with water (6.2 mL), which was added over 45 minutes at 2° C. to form the dichloride vinamidinium salt. After stirring for 1 h, the deposited solid was collected and washed with isopropyl acetate (2×14 mL) and acetonitrile (2×14 mL) to afford the dichloride vinamidinium salt (12.0 g, 54%) as a pale yellow crystal. To a slurry mixture of dichloride vinamidinium salt (10.1 g, 39.9 mmole) and acetamidine hydrochloride (4.2 g, 44.4 mmol) in acetonitrile (48 mL) at 22° C. was added 50% sodium hydroxide (4.9 g, 61.1 mmol) over 1.5 h and stirred at room temperature for 1.5 h. The reaction mixture was filtered and washed with acetonitrile (10 mL), and the combined filtrate was concentrated under reduced pressure and solvent switched to heptane. The resulting heptane slurry mixture of crude 2-6 (25 mL) was extracted with methyl t-butyl ether (MTBE) (4×20 mL) at 40° C. The combined MTBE extract was filtered through a pad of fine silica gel and concentrated under reduced pressure. The residue was recrystallized from heptane to give aldehyde 2-6 (2.15 g) as pale yellow solid; m.p. 78-79° C.

¹H NMR (400.25 MHz, CDCl₃): δ10.09 (s, 1H), 9.03 (s, 2H), 2.79 (s, 3H) ppm.

¹³C NMR (100.64 MHz, CDCl₃): δ189.0, 173.2, 158.2, 126.3, 26.7 ppm.

### 1-(2-Methylpyrimidin-5-yl)-7-(5,6,7,8-tetrahydro-[1,8]naphthyridin-2-yl)-hept-1-en-3-one (2-7)

A stirred suspension of anhydrous powdered K₂CO₃ (325 mesh; 6.21 g, 45 mmol), ketophosphonate 2-5 (7.66 g, 22.5 mmol), and 2-methyl-pyrimidine-5-carboxaldehyde (2-6) (2.5 g, 20.5 mmol) in THF (250 mL) was heated at reflux for 4 h. After cooling to room temperature, the mixture was diluted with EtOAc (500 mL) and washed with water (100 mL) and brine (100 mL). The organic solution was dried over MgSO₄, filtered and concentrated. The residue was purified by flash chromatography (SiO2; 10% EtOH/CH₂Cl₂) to give 5.66 g of the enone adduct 2-7 as a tan solid.

¹H NMR (400 MHz, CDCl₃): δ8.7 (s, 2H), 7.39 (d, J=16 Hz, 1H), 7.02 (d, J=7.2 Hz, 1H), 6.77 (d, J=16 Hz, 1H), 6.31 (d, J=7.2 Hz, 1H) 4.81 (br s, 1H), 3.36 (m, 2H), 2.73 (s, 3H), 2.65 (m, 4H), 2.55 (m, 2H), 1.86 (m, 2H), 1.70 (m, 4H) ppm

### 1-(2-Methylpyrimidin-5-yl)-7-(5,6,7,8-tetrahydro-[,1,8]naphthyridin-2-yl)-hept-1-en-3-ol (2-8)

Achiral Reduction

Enone 2-7 (7.13 g, 22.0 mmol) was dissolved in MeOH (200 mL) and cooled to 0° C. Solid NaBH₄ (1.00 g, 1.2 eq) was added in 3 portions. After 15 min of stirring, TLC indicated complete reaction. The reaction was quenched with 10% aqueous citric acid and 1N aqueous HCl was added to effect pH 3. The mixture was stirred for 20 min and then aqueous 1N NaOH was added to effect a pH 9. Methanol was removed in vacuo and the residue was extracted with CHCl₃ (3×100 mL). The combined organic solutions were dried over MgSO₄, filtered, and concentrated. The residue was purified by flash chromatography (SiO₂; 80%/10%/10% CHCl₃/MeOH/EtOAc) to give 6.10 g of the racemic allylic alcohol as a pale yellow solid. The enantiomerically enriched R-isomer 2-8 was isolated by chiral preparative HPLC (10×50 cm Chiralpak AD column, 80/20 EtOH/Hexanes+0.1% diethylamine; 6.1 g injection @ 300 mL/min) to give 2.72 g of the first eluting R-isomer (R_{T}=40 to 51 min; Enantiomer I; >98% ee) and 2.1 g of the second eluting S-isomer (R_{T}=51 to 62 min; Enantiomer II).

Chiral Reduction

A solution of (S)-2-methyl-CBS-oxazoborolidine (OAB-borane) (39.16 mL, 1.0 M in PhMe) in dichloromethane (100 mL) was treated with borane-DMS (4.03 mL, 40.7 mmol). The clear yellow solution was stirred at room temperature for 45 min. In a separate flask, a solution of the enone 2-7 (10.53 g) in CH₂Cl₂ (250 mL) was cooled to −40° C. and treated with triethylamine (4.27 mL, 31.33 mmol). To this cooled solution was added via cannula a solution of the (S)-OAB-borane complex. After stirring for 1 h at −40° C., methanol was added to quench the excess (S)-OAB-borane. The reaction was stirred for 12 h with warming to room temperature. The solution was concentrated to an oil which was purified by flash chromatography (SiO₂; 10% EtOH/CH₂Cl₂) to give 7.9 g of product (15:1 ratio of enantiomers: Chiralpak AD 4.6×250 mm column, 80/20 EtOH/hexanes, 0.8 mL/min) contaminated with diphenylprolinol. This residue was further purified by chiral preparative HPLC 10×50 cm Chiralpak AD column, 80/20 EtOH/Hexanes+0.1% diethylamine; 7.9 g injection @ 300 mL/min) to give 3.5 g of pure 2-8 as a tan solid.

¹H NMR (400 MHz, CDCl₃): δ8.58 (s, 2H), 7.02 (d, J=7.2 Hz, 1H), 6.47 (d, J=16 Hz, 1H), 6.30 (dd, J=5.6, 16 Hz, 1H), 6.29 (d, J=7.2 Hz, 1H), 4.94 (br s, 1H), 4.33 (dt, J=2.0, 6.0 Hz, 1H), 3.34 (m, 2H), 2.68 (s, 3H), 2.62 (t, J=6.4 Hz, 2H), 2.54 (t, J=7.6 Hz, 2H), 1.85 (m, 2H), 1.67 (m, 4H), 1.46 (m, 2H) ppm.

### 3S-(2-Methylpyrimidin-5-yl)-9-(5,6,7,8-tetrahydro-[1,8]naphthyridin-2-yl)-non4-enoic acid ethyl ester (2-9)

To a stirred solution of allylic alcohol 2-8 (4.7 g, 13.9 mmol) in triethylorthoacetate (88 mL) was added a solution of propionic acid (5.34 mL of a 0.15 M soln in (EtO)₃CMe; 0.80 mmol). The solution was heated to reflux (145 ° C.) and reaction progress monitored by HPLC. After 120 min at reflux, the solution was cooled to room temperature and the reaction treated with 1N HCl/brine (25 mL/25 mL). After stirring for 10 min, the mixture was neutralized and extracted with EtOAc (3×100 mL) and the combined organic solutions dried over MgSO₄, filtered and concentrated. The residue was purified by flash chromatography (SiO₂; 80% CHCl₃/10% EtOAc/10% MeOH) to give 4.6 g of 2-9 as a brown oil.

HPLC R_{T}=10.5 min (Vydac C18; 95/5 to 5/95 H₂O/MeCN gradient over 15 min).

¹H NMR (300 MHz, CDCl₃): ≢8.52 (s, 2H), 7.04 (d, J=7.3 Hz, 1H), 6.34 (d, J=7.3 Hz, 1H), 5.53 (m, 2H), 5.05 (br s, 1H), 4.07 (q, J=7.0 Hz, 2H), 3.81 (m, 1H), 3.49 (s, 3H), 2.69 (m, 3H), 2.58 (m, 3H), 2.05 (m, 2H), 1.90 (m, 2H), 1.63 (m, 2H), 1.41 (m, 2H), 1.18 (t, J=7.0 Hz, 3H) ppm.

### 3S-(2-Methylpyrimidin-5-yl)-9-(5,6,7,8-tetrahydro-[1,8]naphthyridin-2-yl)-nonanoic acid ethyl ester (2-10)

Unsaturated ester 2-9 (12.3 g, 30.1 mmol) was dissolved in ethanol (300 mL) and the solution purged with argon gas for 20 min. 10% Palladium on carbon (3.2 g) was added. A balloon of hydrogen gas was affixed to the partially evacuated flask. The heterogeneous reaction was stirred and reaction progress monitored by HPLC. After 3.5 h, the reaction mixture was filtered through Celite, and the filtrate concentrated. The residue was purified by flash chromatography (SiO₂; 80% CHCl₃/10% MeOH/10% EtOAc) to give 10.2 g of ester 2-10 as a colorless oil.

HPLC R_{T}=10.6 min (Vydac C18; 95/5 to 5/95 H₂O/MeCN gradient over 15 min).

¹H NMR (400 MHz, CDCl₃): 8.43 (s, 2H), 7.04 (d, J=7.2 Hz, 1H), 6.27 (d, J=7.2 Hz, 1H), 4.01 (q, J=7.1 Hz, 2H), 3.37 (m, 2H), 3.01 (m, 1H), 2.64 (m, 3H), 2.68 (s, 3H), 2.48 (m, 3H), 1.87 (m, 2H), 1.59 (m, 3H), 1.24 (m, 3H), 1.12 (t, J=7.1 Hz, 2H) ppm.

### 3S-(2-Methylpyrimidin-5-yl)-9-(5,6,7,8-tetrahydro-[1,8]naphthyridin-2-yl)-nonanoic acid (2-11)

To a stirred solution of ester 2-10 (10.2 g, 24.8 mmol) in MeOH/THF (50 mL/150 mL) was added 1N NaOH (75 mL, 75 mmol). The reaction was stirred for 16 h at rt and then neutralized with 1N HCl (75 mL). The solvent was removed in vacuo. The residue was purified by flash chromatography (SiO₂; 80% CHCl₃/10% MeOH/10% EtOAc) to give a viscous foam which was redissolved in a minimal volume of water to produce a white gummy paste. Stirring and scratching with a metal spatula produced a white crystalline solid. The precipitate was collected by filtration to give 8.0 g of 2-11.

HPLC R_{T}=9.20 min (Vydac C18; 95/5 to 5/95 H₂O/MeCN gradient over 15 min).

¹H NMR (400 MHz, CD₃OD): δ8.54 (s, 2H), 7.36 (d, J=7.2 Hz, 1H), 6.43 (d, J=7.2 Hz, 1H), 3.39 (t, J=8.0 Hz, 2H), 3.27 (s, 3H), 3.12 (m, 1H), 2.72 (t, J=6.0 Hz, 2H), 2.58 (m, 4H), 1.87 (m, 2H), 1.76 (m, 1H), 1.61 (m, 3H), 1.41 (m, 1H), 1.29 (m, 5H) ppm.

### 3(S)-(2-Methyl-pyrimidin-5-yl)-9-[5(R or S)-hydroxy-5,6,7,8-tetrahydro-[1,8]naphthyridin-2-yl]-nonanoic acid (2-12)

Incubation was carried as in Example 1 above, but using 3(S)-(2-methyl-pyrimidin-5-yl)-9-(5,6,7,8-tetrahydro[1,8]naphthyridin-2-yl)-nonanoic acid (2-11) as substrate.

The following compounds were also identified as metabolites of compound 2-11 by means of comparison of their liquid chromatographic mass spectra and NMR spectra with those of authentic materials prepared by unambiguous chemical synthesis detailed below.

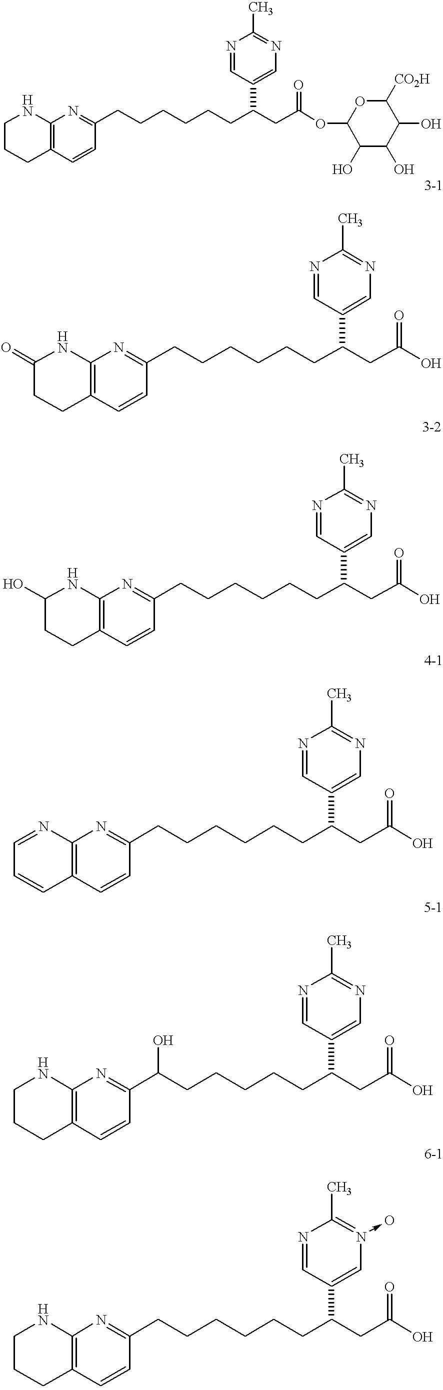

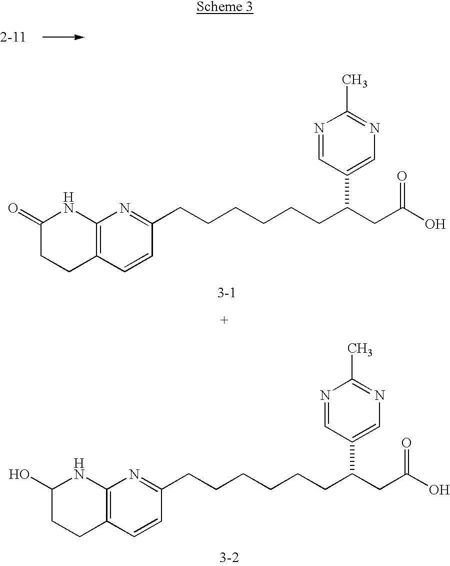

### EXAMPLE 3

### 3S-(2-Methylpyrimidin-5-yl)-9-(7-oxo-5,6,7,8-tetrahydro-[1,8]naphthyridin-2-yl)-8-acetoxy-nonanoic acid (3-1) and 3-(2-methylpyrimidin-5-yl)-9-(7-hydroxy-5,6,7,8-tetrahydro-[1,8]naphthyridin-2-yl)-8-acetoxy-nonanoic acid (3-2)

To a stirred solution of acid 2-11 (0.250 g, 0.65 mmol) in water (4 mL) was added aqueous LiOH (0.65 mL of a 1N aq soln). To this solution was added KMnO₄ (0.103 g, 0.65 mmol) and the reaction stirred at room temperature for 12 h. The reaction was filtered and the filtrate concentrated. The residue was purified by preparative HPLC (Delta-Pak C-18, MeCN/H₂O/0.05% NH₄HCO₃; 0 to 40% MeCN over 60 min; flow=60 mL/min) to give the 7-oxo derivative 3-1 (R_{T}=17.1 min) and the 7-hydroxy derivative 3-2 (R_{T}=17.9 min).

Data for 3-1:¹H NMR (500 MHz, CD₃OD): 67 8.67 (s, 2H), 7.63 (d, J=7.6 Hz, 1H), 6.95 (d, J=7.6 Hz, 1H), 3.11 (m, 1H), 2.97 (t, J=7.7 Hz, 2H), 2.69 (m, 9H) 1.71 (m, 4H), 1.33 (m, 511), 1.18 (m, 1H) ppm.

Data for 3-2 (Major Isomer): ¹H NMR (500 MHz, CD₃OD): δ8.58 (s, 2H), 7.31 (d, J=7.6 Hz, 1H), 6.51 (d, J=7.6 Hz, 1H), 4.69 (s, 1H), 3.13 (m, 2H), 2.82 (m, 2H), 2.65 (m, 7H), 2.10 (m, 1H), 1.75 (m, 1H), 1.61 (m, 5H), 1.28 (m, 3H) ppm.

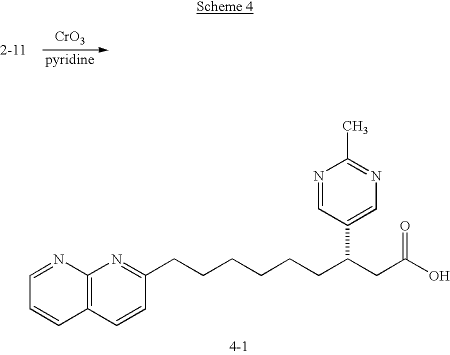

### EXAMPLE 4

### 3S-(2-Methylpyrimidin-5-yl)-9-([1,8]naphthyridin-2-yl)-nonanoic acid (4-1)

To a stirred solution of acid 2-11 (0.2 g, 0.52 mmol) in pyridine (2 mL) at room temperature was added chromium trioxide (0.052 g, 0.52 mmol). After stirring for 12 h, the reaction was concentrated and the residue purified by preparative HPLC (Waters PrepPak, H₂O/MeCN/0.1% TFA gradient 95/5 to 5/95 over 40 min) to give the desired product (0.055 g ) 4-1 as the TFA salt.

HRMS (M+1) calcd 379.2134, found 379.2133.

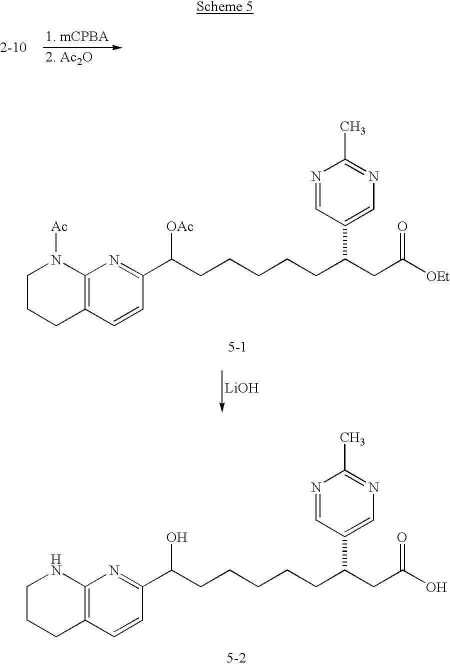

### EXAMPLE 5

### 3S-(2-Methylpyrimidin-5-yl)-9-(8-acetyl-5,6,7,8-tetrahydro-[1,8]naphthyridin-2-yl)-8-acetoxy-nonanoic acid (5-1)

To a stirred solution of ester 2-10 (2.0 g, 4.88 mmol) in dichloromethane (25 mL) was added m-chloroperbenzoic acid (1.32 g) at room temperature. After stirring 12 h, the reaction was further diluted with dichloromethane (40 mL) and washed with water (6×30 mL), saturated aqueous NaHCO₃ (30 mL), and brine (30 mL). The organic solution was dried over Na₂SO₄, filtered and concentrated. The residue was purified by column chromatography (3 to 5% MeOH/CHCl₃) to give 0.870 g of the N-oxide as an oil. The N-oxide was dissolved in acetic anhydride (10 mL) and heated to 90° C. for 12 h. The solution was cooled to room temperature and poured onto ice (ca. 150 g) and neutralized by the addition of solid NaHCO₃. The resulting mixture was extracted with EtOAc (3×75 mL) and the combined organic extracts washed with brine (50 mL), dried over Na₂SO₄, filtered and concentrated. The residue was purified by column chromatography (2% MeOH/CHCl₃) to give the desired product (0.57 g) as a yellow oil.

¹H NMR (500 MHz, CDCl₃): δ8.48 (s, 2H), 7.40 (d, J=7.5 Hz, 1H), 6.96 (d, J=7.5 Hz, 1H), 5.64 (t, J=6.5 Hz, 1H), 4.05 (m, 2H), 3.91 (m, 2H), 3.05 (m, 1H), 2.75 (m, 3H), 2.71 (s, 3H), 2.54 (m, 1H), 2.48 (s, 1H), 2.10 (s, 3H), 1.93 (m, 1H), 1.86 (m, 1H), 1.26 (m, 2H), 1.16 (t, J=7.1 Hz, 2H) ppm.

### 3S-(2-Methylpyrimidin-5-yl)-9-(5,6,7,8-tetrahydro-[1,8]naphthyridin-2-yl)-8-hydroxy-nonanic acid (5-2)

To a stirred solution of 5-1 (0.57 g, 1.12 mmol) in ethanol (5.6 mL) was added aqueous NaOH (5.6 mL of a 1N soln, 5.6 mmol) and the reaction stirred for 12 h at rt. The reaction was neutralized with 1N HCl and the solvent removed in vacuo. The residue was purified by preparative HPLC (Waters PrepPak, H₂O/MeCN/0.1% TFA gradient 95/5 to 5/95 over 40 min) to give the desired product 5-2 as the TFA salt.

¹H NMR (500 MHz, CD₃OD): δ8.59 (s, 2H), 7.62 (d, J=7.3 Hz, 1H), 6.66 (d, J=7.3 Hz, 1H), 4.67 (m, 2H), 4.05 (m, 2H), 3.51 (m, 2H), 3.09 (m, 1H), 2.83 (m, 2H), 2.74 (m, 1H), 2.66 (s, 3H), 2.64 (m, 1H), 1.96 (m, 2H), 1.75 (m, 1H), 1.68 (m 3H), 1.36 (m, 5H), 1.10 (m, 1H) ppm.

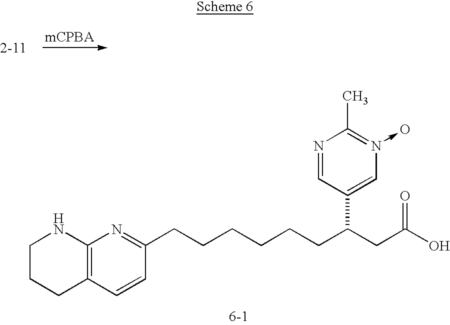

### EXAMPLE 6

### (3S)-3-(2-methyl-1-oxidopyrimidin-5-yl)-9-(5 ,6,7,8-tetrahydro-1,8-naphthyridin-2-yl)nonanoic acid (6-1)

To a stirred solution of the acid 2-11 (0.5 g, 1.31 mmol) in dichloromethane (6 mL) was added m-chloroperbenzoic acid (0.33 g) at room temperature. After stirring 16 h, the reaction was diluted with dichloromethane (50 mL) and washed with water (6×30 mL), satd aq NaHCO₃ (30 mL), and brine (30 mL). The organic solution was dried over Na₂SO₄, filtered and concentrated. The residue was purified by column chromatography (95/5 CHCl₃/MeOH to 70/15/15 CHCl₃/MeOH/EtOAc) to give 0.06 g of the N-oxide as a white solid.

HPLC R_{T}=9.5 min (Vydac C18; 95/5 to 5/95 H₂O/MeCN gradient over 15 min).

HMS (M+1) calcd 399.2391, found 399.2393.

¹H NMR (500 MHz, CD₃OD): δ8.51 (d, J=2.0 Hz, 1H), 8.32 (d, J=2.0 Hz, 1H), 7.41 (d, J=7.3 Hz, 1H), 6.49 (d, J=7.3 Hz, 1H), 3.44 (t, J=5.6 Hz, 2H), 3.16 (m, 1H), 2.77 (t, J=6.1 Hz, 2H), 2.66 (s, 3H), 2.60 (m, 3H), 2.52 (dd, J=8.1, 13 Hz, 1H), 1.92 (m, 2H), 1.77 (m, 1H), 1.64 (m, 3H), 1.43 (m, 1H), 1.32 (m, 4H), 1.24 (m 1H) ppm.

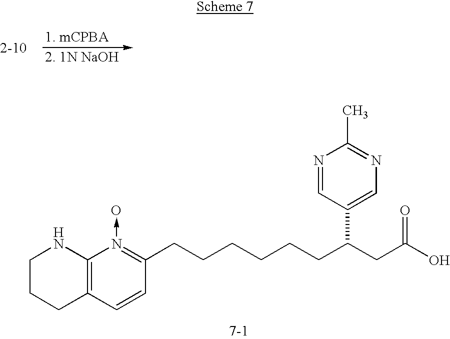

### EXAMPLE 7

### (3S)-3-(2-methylpyrimidin-5-yl)-9-(1-oxido-5,6,7,8-tetrahydro-1,8-naphthyridin-2-yl)nonanoic acid (7-1)

To a stirred solution of ester 2-10 (1.0 g, 2.44 mmol) in dichloromethane (12 mL) was added m-chloroperbenzoic acid (0.66 g) at room temperature. After stirring 12 h, the reaction was further diluted with dichloromethane (100 mL), and washed with water (6×50 mL), saturated aqueous NaHCO₃ (50 mL), and brine (50 mL). The organic solution was dried over Na₂SO₄, filtered and concentrated. The residue was purified by column chromatography (3 to 5% MeOH/CHCl₃) to give 0.25 g as a white solid. The ester was dissolved in MeOH/THF (3 mL5 mL) and 1N NaOH (1.76 mmol, 1.76 mL) was added at room temperature. After stirring for 16 h, the reaction was neutralized with 1N HCl (1.76 mmol, 1.76 mL). The solvent was removed in vacuo, and the residue was purified by preparative HPLC (Waters PrepPak, H₂O/MeCN/0.1% TFA gradient 95/5 to 5/95 over 50 min) to give the desired product 7-1 (0.11 g) as the TFA salt.

HPLC R_{T}=9.7 min (Vydac C18; 95/5 to 5/95 H₂O/MeCN gradient over 15 min).

¹H NMR (500 MHz, CD₃OD): δ8.60 (s, 2H), 7.43 (d, J=7.6 Hz, 1H), 6.60 (d, J=7.6 Hz, 1H), 3.55 (t, J=5.6 Hz, 2H), 3.09 (m, 1H), 2.85 (t, J=6.0 Hz, 2H) 2,79 (t, J=7.7 Hz, 2H), 2.75 (dd, J=5.9, 16 Hz, 1H), 2.66 (s, 3H), 2.62 (dd, J=9.4, 16 Hz, 1H), 1.95 (m, 2H), 1.77 (m, 1H), 1.66 (m, 3H), 1.36 (m, 4H), 1.26 (m, 1H), 1.16 (m, 1H) ppm.

Assays for Determining Biological Activity

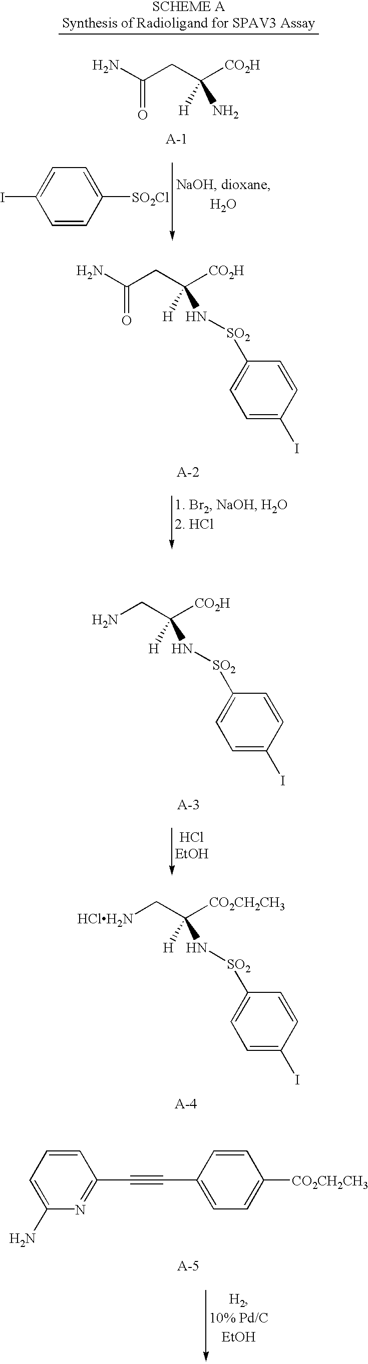

### N-(4-Iodo-phenylsulfonylamino)-L-asparagine (A-2)

To a stirred solution of acid A-1 (4.39 g, 33.2 mmol), NaOH (1.49 g, 5 37.2 mmol), dioxane (30 ml) and H₂O (30 ml) at 020 C. was added pipsyl chloride (10.34 g, 34.2 mmol). After ˜5 minutes, NaOH (1.49, 37.2 mmol) dissolved in 15 ml H₂O, was added followed by the removal of the cooling bath. After 2.0 h, the reaction mixture was concentrated. The residue was dissolved in H₂O (300 ml) and then washed with EtOAc. The aqueous portion was cooled to 0° C. and then acidified with concentrated IICl. The solid was collected and then washed with Et₂O to provide acid A-2 as a white solid.

¹H NMR (300 MHz, D₂O) δ7.86 (d, 2H, J=8 Hz ), 7.48 (d, 2H, J=8 Hz) 3.70 (m,1H), 2.39 (m, 2H).

### 2(S)-(4-Iodo-phenylsulfonylamino)-β-alanine (A-3)

To a stirred solution of NaOH (7.14 g, 181.8 mmol) and H₂O (40 ml) at 0° C. was added Br₂ (1.30 ml, 24.9 mmol) dropwise over a ten minute period. After ˜5 minutes, acid A-2 (9.9 g, 24.9 mmol), NaOH (2.00 g, 49.8 mmol) and H₂O (35 ml) were combined, cooled to 0° C. and then added in a single portion to the reaction. After stirring for 20 minutes at 0° C., the reaction was heated to 90° C. for 30 minutes and then recooled to 0° C. The pH was adjusted to ˜7 by dropwise addition of concentrated HCl. The solid was collected, washed with EtOAc, and then dried in vacuo to provide acid A-3 as a white solid.

¹H NMR (300 MHz, D₂O) δ8.02 (d, 2H, J=8 Hz), 7.63 (d, 2H, J=8 Hz), 4.36 (m, 1H), 3.51 (dd, 1H, J=5 Hz, 13 Hz) 3.21 (m, 1H).

### Ethyl 2(S)-(4-iodo-phenylsulfonylamino)-β-alanine-hydrochloride (A-4)

HCl gas was rapidly bubbled through a suspension of acid A-3 (4.0 g, 10.81 mmol) in EtOH (50 ml) at 0° C. for 10 minutes. The cooling bath was removed and the reaction was heated to 60° C. After 18 h, the reaction was concentrated to provide ester A-4 as a white solid.

¹H NMR (300 MHz, CD₃OD) δ7.98 (d, 2H, J=8 Hz), 7.63 (d, 2H, J=8 Hz), 4.25 (q, 1H, J=5 Hz), 3.92 (m, 2H), 3.33 (m, 1H), 3.06 (m, 1H), 1.01 (t, 3H, J=7 Hz).

### Ethyl 4-[2-(2-Aminopyridin-6-yl)ethyl]benzoate (A-5a)

A mixture of ester A-5 (700 mg, 2.63 mmol), (for preparation, see: Scheme 29 of PCT International Application Publication No. WO 95/32710, published Dec. 7, 1995) 10% Pd/C (350 mg) and EtOH were stirred under 1 atm H₂. After 20 h, the reaction was filtered through a celite pad and then concentrated to provide ester A-5a as a brown oil.

TLC Rf=0.23 (silica, 40% EtOAc/hexanes)

¹H NMR (300 MHz, CDCl3) δ7.95 (d, 2H, J=8 Hz), 7.26 (m, 3H), 6.43 (d, 1H, J=7 Hz), 6.35 (d, 1H, J=8 Hz), 4.37 (m, 4H), 3.05 (m, 2H), 2.91 (m, 2H), 1.39 (t, 3H, J=7 Hz).

### 4-[2-(2-Aminopyridin-6-yl)ethyl]benzoic acid hydrochloride (A-6)

A suspension of ester A-5a (625 mg, 2.31 mmol) in 6N HCl (12 ml) was heated to 60° C. After ˜20 h, the reaction was concentrated to give acid A-6 as a tan solid.

¹H NMR (300 MHz, CD₃OD) δ7.96 (d, 2H, J=8 Hz), 7.80 (m, 1H), 7.33 (d, 2H, J=8 Hz), 6.84 (d, 1H, J=9 Hz), 6.69 (d, 1H, J=7 Hz), 3.09 (m, 4H).

### Ethyl 4-[2-(2-Aminopyridin-6-yl)ethyl]benzoyl-2(S)-(4-iodo-phenylsulfonylamino)-β-alanine (A-7)

A solution of acid 15-6 (400 mg, 1.43 mmol), amine A-4 (686 mg, 1.57 mmol), EDC (358 mg, 1.86 mmol), HOBT (252 mg, 1.86 mmol), NMM (632 μl, 5.72 mmol) in DMF (10 ml) was stirred for ˜20 h. The reaction was diluted with EtOAc and then washed with sat. NaHCO₃, brine, dried (MgSO4) and concentrated. Flash chromatography (silica, EtOAc then 5% isopropanol/EtOAc) provided amide A-7 as a white solid.

TLC R_{f}=0.4 (silica, 10% isopropanol/EtOAc)

¹H NMR (300 MHz, CD₃OD) δ7.79 (d, 2H, J=9 Hz) 7.61 (d, 2H, J=8 Hz), 7.52 (d, 2H, J=9 Hz), 7.29 (m, 1H), 7.27 (d, 2H, J=8 Hz), 4.20 (m, 1H), 3.95 (q, 2H), 2.86 (m, 2H), 3.66 (dd, 1H, J=6 Hz, 14 Hz), 3.49 (dd, 1H, J=8 Hz, 13 Hz), 3.01 (m, 2H), 2.86 (m, 2H), 1.08 (t, 3H, J=7 Hz).

### 4-[2-(2-Aminopyridin-6-yl)ethyl]benzoyl-2(S)-(4-iodophenyl-sulfonylamino)-β-alanine (A-8)

A solution of ester A-7 (200 mg, 0.3213 mmol) and 6N HCl (30 ml) was heated to 60° C. After ˜20 h, the reaction mixture was concentrated. Flash chromatography (silica, 20:20:1:1 EtOAc/EtOH/NH₄OH/H₂O) provided acid A-8 as a white solid.

TLC R_{f}=0.45 (silica, 20:20:1:1 EtOAc/EtOH/NH₄OH/H₂O)

¹H NMR (400 MHz, DMSO) δ8.40 (m, 1H), 8.14 (Bs, 1H), 7.81 (d, 2H, J=8 Hz), 7.62 (d, 2H, J=8 Hz), 7.48 (d, 2H, J=8 Hz), 7.27 (m, 3H), 6.34 (d, 1H, J=7 Hz), 6.25 (d, 1H, J=8 Hz), 5.85 (bs, 2H), 3.89 (bs, 1H), 3.35 (m, 2H), 2.97 (m, 2H), 2.79 (m, 2H).

### 4-[2-(2-Aminopyridin-6-yl)ethyl)benzoyl-2(S)-(4-trimethylstannyl-phenylsulfonylamino-β-alanine (A-9)

A solution of iodide A-8 (70 mg, 0.1178 mmol), [(CH₃)₃Sn]₂ (49 μl, 0.2356 mmol), Pd(PPh₃)₄ (5 mg) and dioxane (7 ml) was heated to 90° C. After 2 h, the reaction was concentrated and then purified by preparative HPLC (Delta-Pak C₁₈15 μM 100A°, 40×100 mm; 95:5 then 5:95 H₂O/CH₃CN) to provide the trifluoroacetate salt. The salt was suspended in H₂O (10 ml), treated with NH₄OH (5 drops) and then lyophilized to provide amide A-9 as a white solid.

¹H NMR (400 MHz, DMSO) δ8.40 (m, 1H), 8.18 (d, 1H, J=8 Hz), 7.67 (m, 5H), 7.56 (d, 2H, J=8 Hz), 7.29 (d, 2H, J=8 Hz), 6.95-7.52 (m, 2H), 6.45 (bs, 2H), 4.00 (m, 1H), 3.50 (m, 1H), 3.33 (m, 1H), 2.97 (m, 2H), 2.86 (m, 2H).

### 4-[2-(2-Aminopyridin-6-yl)ethyl]benzoyl-2(S)-4-¹²⁵iodo-phenylsulfonylamino-β-alanine (A-10)

An iodobead (Pierce) was added to a shipping vial of 5 mCi of Na¹²⁵I (Amersham, IMS30) and stirred for five minutes at room temperature. A solution of 0.1 mg of A-9 in 0.05 mL of 10% H₂SO₄/MeOH was made and immediately added to the Na¹²⁵I/iodobead vial. After stirring for three minutes at room temperature, approximately 0.04-0.05 mL of NH₄OH was added so the reaction mixture was at pH 6-7. The entire reaction mixture was injected onto the HPLC for purification [Vydac peptide-protein C-18 column, 4.6×250 mm, linear gradient of 10% acetonitrile (0.1% (TFA):H₂O (0.1% TFA) to 90% acetonitrile (0.1% TFA):H₂O (0.1% TFA) over 30 minutes, 1 mL/min]. The retention time of A-10 is 17 minutes under these conditions. Fractions containing the majority of the radioactivity were pooled, lyophilized and diluted with ethanol to give approximately 1 mCi of A-10, which coeluted on HPLC analysis with an authentic sample of A-8.

Instrumentation: Analytical and preparative HPLC was carried out using a Waters 600E Powerline Multi Solvent Delivery System with 0.1 mL heads with a Rheodyne 7125 injector and a Waters 990 Photodiode Array Detector with a Gilson FC203 Microfraction collector. For analytical and preparative HPLC, a Vydac peptide-protein C-18 column, 4.6×250 mm was used with a C-18 Brownlee modular guard column. The acetonitrile used for the HPLC analyses was Fisher Optima grade. The HPLC radiodetector used was a Beckman 170 Radioisotope detector. A Vydac C-18 protein and peptide column, 3.9×250 mm was used for analytical and preparative HPLC. Solutions of radioactivity were concentrated using a Speedvac vacuum centrifuge. Calibration curves and chemical concentrations were determined using a Hewlett Packard Model 8452A UV/Vis Diode Array Spectrophotometer. Sample radioactivities were determined in a Packard A5530 gamma counter.

The test procedures employed to measure αvβ3 binding and the bone resorption inhibiting activity of the compounds of the present invention are described below.

BONE RESORPTION-PIT ASSAY

When osteoclasts engage in bone resorption, they can cause the formation of pits in the surface of bone that they are acting upon. Therefore, when testing compounds for their ability to inhibit osteoclasts, it is useful to measure the ability of osteoclasts to excavate these resorption pits when the inhibiting compound is present.

Consecutive 200 micron thick cross sections from a 6 mm cylinder of bovine femur diaphysis are cut with a low speed diamond saw (Isomet, Beuler, Ltd., Lake Bluff, Ill.). Bone slices are pooled, placed in a 10% ethanol solution and refrigerated until further use.

Prior to experimentation, bovine bone slices are ultrasonicated twice, 20 minutes each in H₂O. Cleaned slices are placed in 96 well plates such that two control lanes and one lane for each drug dosage are available. Each lane represents either triplicate or quadruplicate cultures. The bone slices in 96 well plates are sterilized by UV irradiation. Prior to incubation with osteoclasts, the bone slices are hydrated by the addition of 0.1 ml αMEM, pH 6.9 containing 5% fetal bovine serum and 1% penicillin/streptomycin.

Long bones from 7-14 day old rabbits (New Zealand White Hare) are dissected, cleaned of soft tissue and placed in αMEM containing 20 mM HEPES. The bones are minced using scissors until the pieces are <1 mm and transferred to a 50 ml tube in a volume of 25 ml. The tube is rocked gently by hand for 60 cycles, the tissue is sedimented for 1 min., and the supernatant is removed. Another 25 ml of medium is added to the tissue and rocked again. The second supernatant is combined with the first. The number of cells is counted excluding erythrocytes (typically ˜2×10⁷ cells/ml). A cell suspension consisting of 5×10⁶/ml in αMEM containing 5% fetal bovine serum, 10 nM 1,25(OH)₂D₃, and pencillin-streptomycin is prepared. 200 ml aliquots are added to bovine bone slices (200 mm×6 mm) and incubated for 2 hrs. at 37° C. in a humidified 5% CO₂ atmosphere. The medium is removed gently with a micropipettor and fresh medium containing test compounds is added. The cultures are incubated for 48 hrs., and assayed for c-telopeptide (fragments of the al chain of type I collagen) by Crosslaps for culture media (Herlev, Denmark).

Bovine bone slices are exposed to osteoclasts for 20-24 hrs and are processed for staining. Tissue culture media is removed from each bone slice. Each well is washed with 200 ml of H₂O, and the bone slices are then fixed for 20 minutes in 2.5% glutaraldehyde, 0.1 M cacodylate, pH 7.4. After fixation, any remaining cellular debris is removed by 2 min. ultrasonication in the presence of 0.25 M NII₄OH followed by 2×15 min ultrasonication in H₂O. The bone slices are immediately stained for 6-8 min with filtered 1% toluidine blue and 1% borax.

After the bone slices have dried, resorption pits are counted in test and control slices. Resorption pits are viewed in a Microphot Fx (Nikon) fluorescence microscope using a polarizing Nikon IGS filter cube. Test dosage results are compared with controls and resulting IC₅₀ values are determined for each compound tested.

The appropriateness of extrapolating data from this assay to mammalian (including human) disease states is supported by the teaching found in Sato, M., et al, Journal of Bone and Mineral Research, Vol. 5, No. 1, pp. 31-40, 1990, which is incorporated by reference herein in its entirety. This article teaches that certain bisphosphonates have been used clinically and appear to be effective in the treatment of Paget's disease, hypercalcemia of malignancy, osteolytic lesions produced by bone metastases, and bone loss due to immobilization or sex hormone deficiency. These same bisphosphonates are then tested in the resorption pit assay described above to confirm a correlation between their known utility and positive performance in the assay.

EIB ASSAY

Duong et al., J. Bone Miner. Res., 8: S378 (1993), describes a system for expressing the human integrin αvβ3. It has been suggested that the integrin stimulates attachment of osteoclasts to bone matrix, since antibodies against the integrin, or RGD-containing molecules, such as echistatin (European Publication 382 451), can effectively block bone resorption.

Reaction Mixture

1. 175 μl TBS buffer (50 mM Tris.HCl pH 7.2, 150 mM NaCl, 1% BSA, 1 mM CaCl₂, 1 mM MgCl₂).

2. 25 ml cell extract (dilute with 100 mM octylglucoside buffer to give 2000 cpm/25 μl).

3. ¹²⁵I-echistatin (25 μl/50,000 cpm) (see EP 382 451).

4. 25 μl buffer (total binding) or unlabeled echistatin (non-specific binding).

The reaction mixture was then incubated for 1 h at room temp. The unbound and the bound αvβ3 were separated by filtration using a Skatron Cell Harvester. The filters (prewet in 1.5% poly-ethyleneimine for 10 mins) were then washed with the wash buffer (50 mM Tris HCl, 1 mM CaCl₂/MgCl₂, pH 7.2). The filter was then counted in a gamma counter.

SPAV3 Assay

Materials

1. Wheat germ agglutinin Scintillation Proximity Beads (SPA): Amersham

2. Octylglucopyranoside: Calbiochem

3. HEPES: Calbiochem

4. NaCl: Fisher

5. CaCl₂: Fisher

6. MgCl₂: SIGMA

7. Phenylmethylsulfonylfluoride (PMSF): SIGMA

8. Optiplate: PACKARD

9. Compound A-10 (specific activity 500-1000 Ci/mmole)

10. test compound

11. Purified integrin receptor: αvβ3 was purified from 293 cells overexpressing αvβ3 (Duong et al., J. Bone Min. Res., 8:S378, 1993) according to Pytela (Methods in Enzymology, 144:475, 1987)

12. Binding buffer: 50 mM HEPES, pH 7.8, 100 mM NaCl, 1 mM Ca²⁺/Mg²⁺, 0.5 mM PMSF

13. 50 mM octylglucoside in binding buffer: 50-OG buffer

Procedure

1. Pretreatment of SPA beads:

500 mg of lyophilized SPA beads were first washed four times with 200 ml of 50-OG buffer and once with 100 ml of binding buffer, and then resuspended in 12.5 ml of binding buffer.

2. Preparation of SPA beads and receptor mixture

In each assay tube, 2.5 μl (40 mg/ml) of pretreated beads were suspended in 97.5 μl of binding buffer and 20 ml of 50-OG buffer. 5 ml (˜30 ng/μl) of purified receptor was added to the beads in suspension with stirring at room temperature for 30 minutes. The mixture was then centrifuged at 2,500 rpm in a Beckman GPR Benchtop centrifuge for 10 minutes at 4° C. The pellets were then resuspended in 50 μl of binding buffer and 25 μl of 50-OG buffer.

3. Reaction

The following were sequentially added into Optiplate in corresponding wells:

(i) Receptor/beads mixture (75 μl)

(ii) 25 μl of each of the following: compound to be tested, binding buffer for total binding or A-8 for non-specific binding (final concentration 1 μM)

(iii) A-10 in binding buffer (25 μl, final concentration 40 pM)

(iv) Binding buffer (125 μl)

(v) Each plate was sealed with plate sealer from PACKARD and incubated overnight with rocking at 4° C.

4. Plates were counted using PACKARD TOPCOUNT

5. % inhibition was calculated as follows:

A=total counts

B=nonspecific counts

C=sample counts

% inhibition=[{(A−B)−(C−B)}/(A−B)]/(A−B)×100

Ocform Assay

Osteoblast-like cells (1.8 cells), originally derived from mouse calvaria, were plated in CORNING 24 well tissue culture plates in UMEM medium containing ribo- and deoxyribonucleosides, 10% fetal bovine serum and penicillin-streptomycin. Cells were seeded at 40,000/well in the morning. In the afternoon, bone marrow cells were prepared from six week old male Balb/C mice as follows:

Mice were sacrificed, tibiae removed and placed in the above medium. The ends were cut off and the marrow was flushed out of the cavity into a tube with a 1 mL syringe with a 27.5 gauge needle. The marrow was suspended by pipetting up and down. The suspension was passed through >100 mm nylon cell strainer. The resulting suspension was centrifuged at 350×g for seven minutes. The pellet was resuspended, and a sample was diluted in 2% acetic acid to lyse the red cells. The remaining cells were counted in a hemacytometer. The cells were pelleted and resuspended at 1×10⁶ cells/mL. 50 μl was added to each well of 1.8 cells to yield 50,000 cells/well and 1,25-dihydroxy-vitamin D₃ (D₃) was added to each well to a final concentration of 10 nM. The cultures were incubated at 37° C. in a humidified, 5% CO₂ atmosphere. After 48 h, the medium was changed. 72 h after the addition of bone marrow, test compounds were added with fresh medium containing D₃ to quadruplicate wells. Compounds were added again after 48 h with fresh medium containing D₃. After an additional 48 h., the medium was removed, cells were fixed with 10% formaldehyde in phosphate buffered saline for 10 minutes at room temperature, followed by a 1-2 minute treatment with ethanol:acetone (1:1) and air dried. The cells were then stained for tartrate resistant acid phosphatase as follows:

The cells were stained for 10-15 minutes at room temperature with 50 mM acetate buffer, pH 5.0 containing 30 mM sodium tartrate, 0.3 mg/mL Fast Red Violet LB Salt and 0.1 mg/iL Naphthol AS-MX phosphate. After staining, the plates were washed extensively with deionized water and air dried. The number of multinucleated, positive staining cells was counted in each well.

Compounds of structural formula (I) of the present invention were tested and found to bind to human αvβ3 integrin. These compounds were found to have IC50 values less than 100 nM in the SPAV3 assay.

### EXAMPLE OF A PHARMACEUTICAL FORMULATION

As a specific embodiment of an oral composition, 100 mgs of Example 1 or Example 2 are formulated with sufficient finely divided lactose to provide a total amount of 580 to 590 mg to fill a size O hard gel capsule.

While the invention has been described and illustrated in reference to certain preferred embodiments thereof, those skilled in the art will appreciate that various changes, modifications and substitutions can be made therein without departing from the spirit and scope of the invention. For example, effective dosages other than the preferred doses as set forth hereinabove may be applicable as a consequence of variations in the responsiveness of the mammal being treated for severity of bone disorders caused by resorption, or for other indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be limited only by the scope of the claims which follow and that such claims be interpreted as broadly as is reasonable.

What is claimed is:

1. A compound of structural formula I: 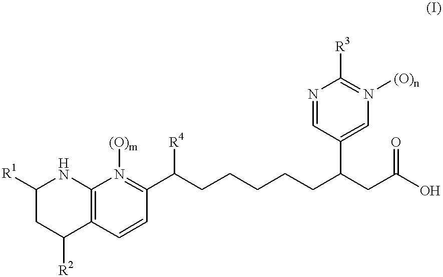
and the individual stereoisomers thereof, or a pharmaceutically acceptable salt thereof; wherein
R¹, R², and R⁴ are each independently hydrogen, hydroxy, or oxo;
R³ is hydrogen or methyl;
m is 0 or 1;
n is 0 or 1;
provided that at least one of R¹, R², and R⁴ is hydroxy or oxo or at least one of n or m is 1;

2. The compound of claim 1 wherein R³ is hydrogen or methyl which is selected from the group consisting of: 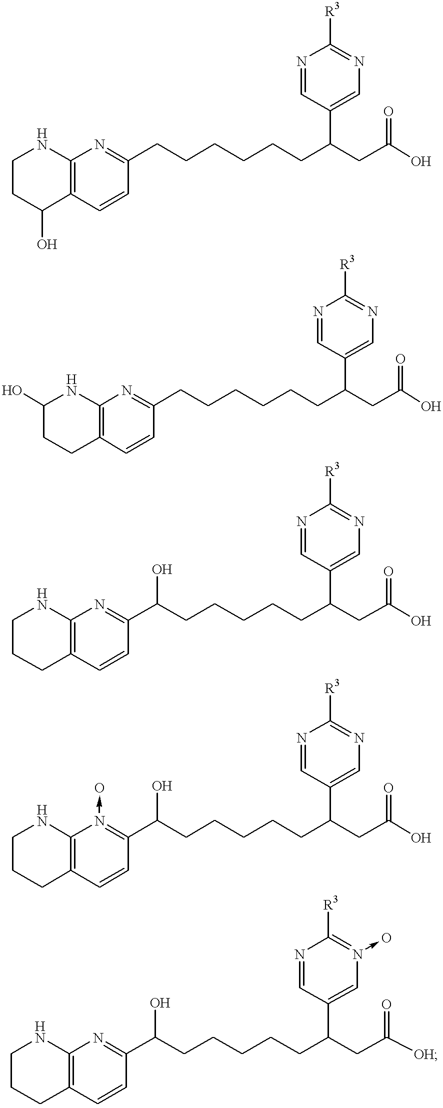
and the individual stereoisomers thereof, or a pharmaceutically salt thereof.

3. The compound of claim 2 wherein R³ is hydrogen or methyl which is selected from the group consisting of: 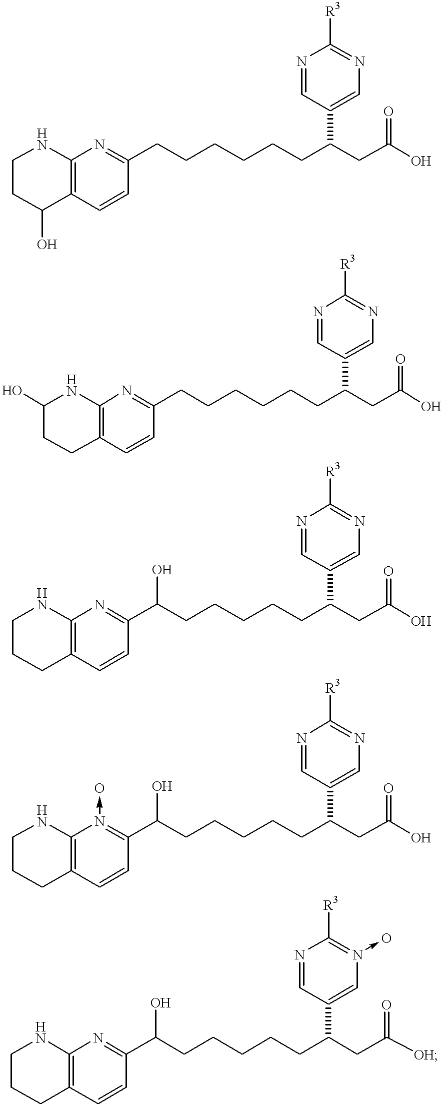
and the individual stereoisomers thereof, or a pharmaceutically salt thereof.

4. A compound of structural formula 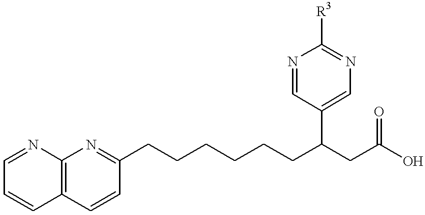
wherein R³ is hydrogen or methyl;
and the individual stereoisomers thereof, or a pharmaceutically salt thereof.

5. The compound of claim 4 which is 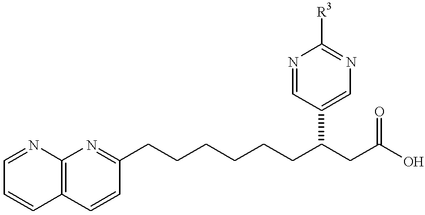
wherein R³ is hydrogen or methyl;
or a pharmaceutically acceptable salt thereof.

6. A pharmaceutical composition comprising a therapeutically effective amount of a compound of claim 1 and a pharmaceutically acceptable carrier.

7. A method of eliciting an αvβ3 integrin receptor antagonizing effect in a mammal in need thereof, comprising administering to said mammal a therapeutically effective amount of a compound of claim 1.

8. The method of claim 7 wherein the αvβ3 integrin receptor antagonizing effect is selected from the group consisting of inhibition of bone resorption, restenosis, angiogenesis, diabetic retinopathy, macular degeneration, inflammatory arthritis, cancer, and metastatic tumor growth.

9. The method of claim 8 wherein the αvβ3 integrin receptor antagonizing effect is inhibition of bone resorption.

10. A method of treating or preventing osteoporosis in a mammal in need thereof, comprising administering to the mammal a therapeutically effective amount of a compound of claim 1.
